(19) United States
(12) Patent Application Publication
(10) Pub. No.: US 2025/0228611 A1
(43) Pub. Date: Jul. 17, 2025

(54) ELECTRIC FIELD APPLICATION FOR SINGLE SHOT CARDIAC ABLATION BY IRREVERSIBLE ELECTROPORATION

(71) Applicant: Boston Scientific Scimed, Inc, Maple Grove, MN (US)

(72) Inventors: Brendan E. Koop, Ham Lake, MN (US); Andrew L. De Kock, Ham Lake, MN (US); Allan C. Shuros, St Paul, MN (US)

(21) Appl. No.: 19/172,370

(22) Filed: Apr. 7, 2025

Related U.S. Application Data
(63) Continuation of application No. 17/381,953, filed on Jul. 21, 2021, now Pat. No. 12,268,437.
(60) Provisional application No. 63/056,017, filed on Jul. 24, 2020.

(51) Int. Cl. A61B 18/14; A61B 18/00; A61B 18/12

(52) U.S. Cl. CPC A61B 18/1492; A61B 18/1206; A61B 2018/00577; A61B 2018/00613; A61B 2018/1253; A61B 2018/126; A61B 2018/1266; A61B 2018/1467

(57) ABSTRACT

Disclosed herein are apparatus, systems, and methods for ablating tissue in a patient by electroporation. Embodiments generally include an ablation catheter having a hand, a shaft, and an electroporation electrode arrangement. The shaft has a distal end and defines a longitudinal axis of the ablation catheter. The electroporation electrode arrangement is at the distal end of the shaft and is configured to generate a multidirectional electric field when at least one pulse sequence is delivered thereto. The multidirectional electric field includes at least two of the following directions relative to the longitudinal axis: generally axial, circumferential, and transverse. The electroporation electrode arrangement is configured to operatively couple to an electroporation generator that is configured to generate the at least one pulse sequence and is configured to receive the at least one pulse sequence from the electroporation generator.

### CROSS REFERENCE TO RELATED APPLICATION

This application is a continuation of U.S. patent application Ser. No. 17/381,953, filed Jul. 21, 2021, which claims priority to Provisional Application No. 63/056,017, filed Jul. 24, 2020, both of which are herein incorporated by reference in their entirety.

### TECHNICAL FIELD

The present disclosure relates to medical apparatus, systems, and methods for ablating tissue in a patient. More specifically, the present disclosure relates to medical apparatus, systems, and methods for ablation of tissue by electroporation.

### BACKGROUND

Ablation procedures are used to treat many different conditions in patients. Ablation may be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electric field is applied to cells to increase the permeability of the cell membrane. The electroporation may be reversible or irreversible, depending on the strength of the electric field. If the electroporation is reversible, the increased permeability of the cell membrane may be used to introduce chemicals, drugs, and/or deoxyribonucleic acid (DNA) into the cell, prior to the cell healing and recovering. If the electroporation is irreversible, the affected cells are killed through apoptosis.

Irreversible electroporation may be used as a nonthermal ablation technique. In irreversible electroporation, trains of short, high voltage pulses are used to generate electric fields that are strong enough to kill cells through apoptosis. In ablation of cardiac tissue, irreversible electroporation may be a safe and effective alternative to the indiscriminate killing of thermal ablation techniques, such as RF ablation and cryoablation. Irreversible electroporation may be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells. But the effectiveness of electroporation depends on exposing targeted tissue to a critical electric field strength, which depends on electrode geometry. This much is true because irreversible electroporation effectiveness depends on cell geometry and/or orientation relative to the generated electric field. And often, electrode geometry is such that an electric field produced by the electrodes has limited (e.g., single) orientations.

### SUMMARY

In Example 1, an electroporation ablation system for treating target tissue, the electroporation ablation system comprising an ablation catheter and an electroporation generator. The ablation catheter includes a shaft defining a longitudinal axis of the ablation catheter, and an electroporation electrode arrangement at a distal end of the shaft and including a plurality of electrodes arranged so as to define a plurality of anode-cathode pairs each configured to generate an electric field when an electrical pulse sequence is delivered thereto. The plurality of anode-cathode pairs includes a first anode-cathode pair arranged so as to generate a first electric field having a first orientation relative to the longitudinal axis when a first electrical pulse sequence is delivered thereto, and
a second anode-cathode pair arranged so as to generate a second electric field having a second orientation relative to the longitudinal axis when a second electrical pulse sequence is delivered thereto. The electroporation generator is operatively coupled to the electroporation electrode arrangement and configured to selectively generate and deliver the first electrical pulse sequence to first anode-cathode pair, and the second electrical pulse sequence to the second anode-cathode pair.

In Example 2, the electroporation ablation system of Example 1, wherein the first orientation is generally aligned with the longitudinal axis, and wherein the second orientation is circumferential about the longitudinal axis.

In Example 3, the electroporation ablation system of Example 1, wherein the first orientation is generally aligned with the longitudinal axis, and wherein the second orientation is transverse to the longitudinal axis.

In Example 4, the electroporation ablation system of Example 1, wherein the first orientation is transverse to the longitudinal axis, and the second orientation is generally circumferential about the longitudinal axis.

In Example 5, the electroporation ablation system of any of Examples 1-4, wherein each of the first and second pulse sequences comprises a plurality of direct current (DC) pulses.

In Example 6, the electroporation ablation system of Example 5, wherein the DC pulses are monophasic pulses, biphasic pulses, or triphasic pulses.

In Example 7, the electroporation ablation system of Example 6, wherein the electroporation generator is configured to generate the DC pulses of each electrical pulse sequence separated by an inter-pulse delay, and wherein at least some of the DC pulses of the second electrical pulse sequence are delivered during a respective inter-pulse delay of the first electrical pulse sequence.

In Example 8, the electroporation system of Example 6, wherein the electroporation generator is configured to generate a plurality of electrical pulse sequence sets having a pause therebetween, wherein each electrical pulse sequence set comprises the first electrical pulse sequence and the second electrical pulse sequence.

In Example 9, the electroporation ablation system of Example 6, wherein DC pulses are biphasic pulses, and the electroporation ablation generator is configured to alternate between delivering the first electrical pulse sequence to the first anode-cathode pair, and the second electrical pulse sequence to the second anode-cathode pair.

In Example 10, the electroporation ablation system of Example 6, wherein each of the DC pulses is a triphasic pulse defined by a first phase having a first phase voltage and a first phase length, a second phase having a second phase voltage and a second phase length, and a third phase having a third phase voltage and a third phase length, wherein the first and third phases have the same polarity and the second phase has an opposite polarity thereto.

In Example 11, the electroporation ablation system of Example 10, wherein the first phase voltage, the first phase length, the second phase voltage, the second phase length, the third phase voltage and the third phase length are selected such that each DC pulse is charge-and-energy balanced.

In Example 12, the electroporation ablation system of Example 11, the first, second and third phase voltages are equal to each other, and wherein the first and third phase lengths are equal to one another and have a duration of one-half of the second phase length.

In Example 13, the electroporation ablation system of Example 11, wherein the first and third phase voltages and phase lengths are equal to each other, and are different from the second phase voltage and the second phase length, respectively.

In Example 14, the electroporation ablation system of Example 10, wherein the first, second and third phase lengths and the first, second and third phase voltages are selected such that each DC pulse is charge imbalanced so as to encourage electrolysis of the target tissue.

In Example 15, the electroporation ablation system of Example 5, wherein each DC pulse of the first electrical pulse sequence has a first voltage and a first pulse length selected to ablate the target tissue by irreversible electroporation, and wherein each DC pulse of the second pulse sequence has a second voltage and a second pulse length selected to create electrolytic byproducts at the target tissue proximate the second anode-cathode pair.

In Example 16, an electroporation ablation system for treating target tissue, the electroporation ablation system comprising an ablation catheter and an electroporation generator. The ablation catheter includes a
a handle, a shaft having a distal end and defining a longitudinal axis of the ablation catheter, and an electroporation electrode arrangement at the distal end of the shaft and including a plurality of electrodes spatially arranged so as generate a plurality of electric fields when an electrical pulse sequence is delivered to selected pairs of the electrodes, the plurality of electric fields including a first electric field having a first orientation relative to the longitudinal axis, and second electric field having a second orientation relative to the longitudinal axis. The electroporation generator is operatively coupled to the electroporation electrode arrangement and configured to selectively generate and deliver the electrical pulse sequences to each selected pair of electrodes.

In Example 17, the electroporation ablation system of Example 16, wherein the first orientation is generally aligned with the longitudinal axis, and wherein the second orientation is circumferential about the longitudinal axis.

In Example 18, the electroporation ablation system of Example 16, wherein the first orientation is generally aligned with the longitudinal axis, and wherein the second orientation is transverse to the longitudinal axis.

In Example 19, the electroporation ablation system of Example 16, wherein the first orientation is transverse to the longitudinal axis, and the second orientation is generally circumferential about the longitudinal axis.

In Example 20, the electroporation ablation system of Example 16, wherein each of the plurality of electrical pulse sequences comprises a plurality of direct current (DC) pulses.

In Example 21, the electroporation ablation system of Example 20, wherein the DC pulses are monophasic pulses, biphasic pulses, or triphasic pulses.

In Example 22, the electroporation ablation system of Example 20, wherein the electroporation generator is configured to generate the DC pulses of each pulse sequence separated by an inter-pulse delay, and wherein at least some of the DC pulses of a first electrical pulse sequence are delivered during a respective inter-pulse delay of a second electrical pulse sequence.

In Example 23, the electroporation system of Example 16, wherein the electroporation generator is configured to generate a plurality of electrical pulse sequence sets having a pause therebetween, wherein each electrical pulse sequence set comprises a plurality of electrical pulse sequences.

In Example 24, the electroporation ablation system of Example 20, wherein the electroporation generator is configured to generate the DC pulses of each electrical pulse sequence separated by an inter-pulse delay, and wherein at least some of the DC pulses of the second electrical pulse sequence are delivered during a respective inter-pulse delay of the first electrical pulse sequence.

In Example 25, the electroporation ablation system of Example 20, wherein DC pulses are biphasic pulses, and the electroporation ablation generator is configured to alternate between delivering the first electrical pulse sequence to the first anode-cathode pair, and the second electrical pulse sequence to the second anode-cathode pair.

In Example 26, an electroporation ablation system for treating target tissue, the electroporation ablation system comprising an ablation catheter including a handle, a shaft having a distal end and defining a longitudinal axis of the ablation catheter, and an electroporation electrode arrangement at the distal end of the shaft and including a plurality of electrodes arranged so as to define a plurality of electrode pairs each configured to generate an electric field when an electrical pulse sequence is delivered thereto, the plurality of electrode pairs including a first electrode pair arranged so as to generate a first electric field having a first orientation relative to the longitudinal axis when a first electrical pulse sequence is delivered thereto; and
a second electrode pair arranged so as to generate a second electric field having a second orientation relative to the longitudinal axis when a second electrical pulse sequence is delivered thereto. The electroporation generator is operatively coupled to the electroporation electrode arrangement and configured to selectively generate and deliver the first electrical pulse sequence to first electrode pair, and the second electrical pulse sequence to the second electrode pair.

In Example 27, the electroporation ablation system of Example 26, wherein the first orientation is generally aligned with the longitudinal axis, and wherein the second orientation is circumferential about the longitudinal axis.

In Example 28, the electroporation ablation system of Example 26, wherein the first orientation is generally aligned with the longitudinal axis, and wherein the second orientation is transverse to the longitudinal axis.

In Example 29, the electroporation ablation system of Example 26, wherein the first orientation is transverse to the longitudinal axis, and the second orientation is generally circumferential about the longitudinal axis.

In Example 30, the electroporation ablation system of Example 26, wherein the first and second electrical pulse sequences each comprise a plurality of biphasic DC pulses, and the wherein the electroporation ablation generator is configured to alternate between delivering the first electrical pulse sequence to the first electrode pair, and the second electrical pulse sequence to the second electrode pair.

In Example 31, a method of generating and delivering a signal to electrodes in an electroporation ablation system, the method comprising delivering a first electrical pulse sequence to a first electrode pair of an electroporation catheter having a longitudinal axis so as to generate a first electric field having a first orientation relative to the longitudinal axis, and delivering a second electrical pulse sequence to a second electrode pair of the electroporation catheter so as to generate a second electric field having a second orientation relative to the longitudinal axis.

In Example 32, the method of Example 31, wherein delivering the first electrical pulse sequence to the first electrode pair and delivering the second electrical pulse sequence to the second electrode pair includes alternatingly delivering, over a time period, the first electrical pulse sequence to the first electrode pair and delivering the second electrical pulse sequence to the second electrode pair to produce a dynamically gyrating electric field that comprises a changing pattern over the time period.

In Example 33, the method of Example 32, wherein the first orientation is generally aligned with the longitudinal axis, and wherein the second orientation is circumferential about the longitudinal axis.

In Example 34, the method of Example 32, wherein the first orientation is generally aligned with the longitudinal axis, and wherein the second orientation is transverse to the longitudinal axis.

In Example 35, the method of Example 32, wherein the first orientation is transverse to the longitudinal axis, and the second orientation is generally circumferential about the longitudinal axis.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient and for treating a heart of the patient, using an electrophysiology system, in accordance with embodiments of the subject matter of the disclosure.

FIG. 2A is a diagram illustrating a distal end of a shaft included in a catheter and interactions between electrode pairs, in accordance with embodiments of the subject matter of the disclosure.

FIG. 2B is a diagram illustrating axial electric fields generated by interactions between electrode pairs, in accordance with embodiments of the subject matter of the disclosure.

FIG. 2C is a diagram illustrating circumferential electric fields generated by interactions between electrode pairs in the catheter, in accordance with embodiments of the subject matter of the disclosure.

FIG. 3A is a diagram illustrating two pulse sequence series that include two pulse sequence sets having pulse sequences with interlaced, monophasic pulses to be delivered to electrodes in an electroporation electrode arrangement of the catheter, in accordance with embodiments of the subject matter of the disclosure.

FIG. 3B is a diagram illustrating two pulse sequence series that include two pulse sequence sets having pulse sequences with interlaced, biphasic pulses to be delivered to electrodes in an electroporation electrode arrangement of the catheter, in accordance with embodiments of the subject matter of the disclosure.

FIGS. 3C-3D are diagrams schematically illustrating exemplary biphasic electrical pulse sequences to be delivered to electrodes in an electrode arrangement according to embodiments of subject matter of the disclosure.

FIG. 3E is a diagram illustrating two pulse sequences, one non-alternating and the other alternating, of charge-and-energy balanced, symmetric triphasic pulses to be delivered to electrodes in an electroporation electrode arrangement of the catheter, in accordance with embodiments of the subject matter of the disclosure.

FIG. 3F is a diagram illustrating two pulse sequences, one non-alternating and the other alternating, of charge-and-energy balanced, asymmetric triphasic pulses to be delivered to electrodes in an electroporation electrode arrangement of the catheter, in accordance with embodiments of the subject matter of the disclosure.

FIGS. 3G-31 are diagrams schematically illustrating exemplary charge-imbalanced pulse sequences to be delivered to delivered to electrodes in an electrode arrangement according to embodiments of subject matter of the disclosure.

FIG. 4 is a diagram showing steps of a method of generating and delivering a signal to electrodes in an electroporation ablation system.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Examples of constructions, materials, and/or dimensions are provided for selected elements. Those skilled in the art will recognize that many of the noted examples have a variety of suitable alternatives.

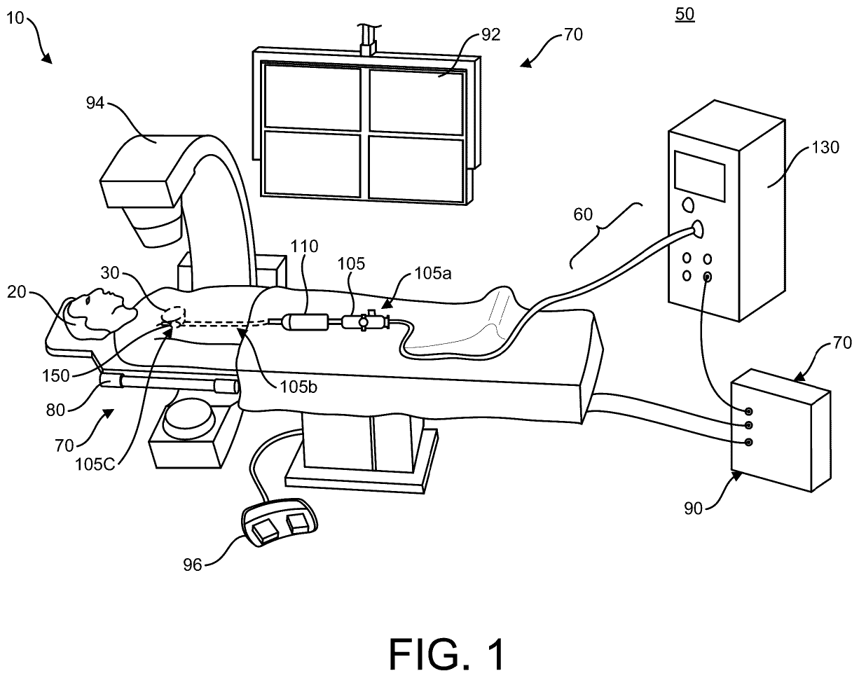

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. The electrophysiology system 50 includes an electroporation catheter system 60 and an electro-anatomical mapping (EAM) system 70, which includes a localization field generator 80, a mapping and navigation controller 90, and a display 92. Also, the clinical setting 10 includes additional equipment such as imaging equipment 94 (represented by the C-arm) and various controller elements, such as a foot controller 96, configured to allow an operator to control various aspects of the electrophysiology system 50. As will be appreciated by the skilled artisan, the clinical setting 10 may have other components and arrangements of components that are not shown in FIG. 1.

The electroporation catheter system 60 includes an electroporation catheter 105, an introducer sheath 110, and an electroporation generator 130. Additionally, the electroporation catheter system 60 includes various connecting elements (e.g., cables, umbilicals, and the like) that operate to functionally connect the components of the electroporation catheter system 60 to one another and to the components of the EAM system 70. This arrangement of connecting elements is not of critical importance to the present disclosure, and one skilled in the art will recognize that the various components described herein may be interconnected in a variety of ways.

In embodiments, the electroporation catheter system 60 is configured to deliver electric field energy to targeted tissue in the patient's heart 30 to create tissue apoptosis, rendering the tissue incapable of conducting electrical signals. The electroporation generator 130 is configured to control functional aspects of the electroporation catheter system 60. In embodiments, the electroporation generator 130 is operable as an electroporation pulse generator for generating and supplying pulse sequences to the electroporation catheter 105, as described in greater detail herein

In embodiments, the electroporation generator 130 includes one or more controllers, microprocessors, and/or computers that execute code out of memory to control and/or perform the functional aspects of the electroporation catheter system 60. In embodiments, the memory may be part of the one or more controllers, microprocessors, and/or computers, and/or part of memory capacity accessible through a network, such as the world wide web.

In embodiments, the introducer sheath 110 is operable to provide a delivery conduit through which the electroporation catheter 105 may be deployed to the specific target sites within the patient's heart 30. It will be appreciated, however, that the introducer sheath 110 is illustrated and described herein to provide context to the overall electrophysiology system 50, but it is not critical to the novel aspects of the various embodiments described herein.

The EAM system 70 is operable to track the location of the various functional components of the electroporation catheter system 60, and to generate high-fidelity three-dimensional anatomical and electro-anatomical maps of the cardiac chambers of interest. In embodiments, the EAM system 70 may be the RHYTHMIA™ HDx mapping system marketed by Boston Scientific Corporation. Also, in embodiments, the mapping and navigation controller 90 of the EAM system 70 includes one or more controllers, microprocessors, and/or computers that execute code out of memory to control and/or perform functional aspects of the EAM system 70, where the memory, in embodiments, may be part of the one or more controllers, microprocessors, and/or computers, and/or part of memory capacity accessible through a network, such as the world wide web.

As will be appreciated by the skilled artisan, the depiction of the electrophysiology system 50 shown in FIG. 1 is intended to provide a general overview of the various components of the system 50 and is not in any way intended to imply that the disclosure is limited to any set of components or arrangement of the components. For example, the skilled artisan will readily recognize that additional hardware components, e.g., breakout boxes, workstations, and the like, may and likely will be included in the electrophysiology system 50.

The EAM system 70 generates a localization field, via the field generator 80, to define a localization volume about the heart 30, and one or more location sensors or sensing elements on the tracked device(s), e.g., the electroporation catheter 105, generate an output that may be processed by the mapping and navigation controller 90 to track the location of the sensor, and consequently, the corresponding device, within the localization volume. In the illustrated embodiment, the device tracking is accomplished using magnetic tracking techniques, whereby the field generator 80 is a magnetic field generator that generates a magnetic field defining the localization volume, and the location sensors on the tracked devices are magnetic field sensors.

In other embodiments, impedance tracking methodologies may be employed to track the locations of the various devices. In such embodiments, the localization field is an electric field generated, for example, by an external field generator arrangement, e.g., surface electrodes, by intra-body or intra-cardiac devices, e.g., an intracardiac catheter, or both. In these embodiments, the location sensing elements may constitute electrodes on the tracked devices that generate outputs received and processed by the mapping and navigation controller 90 to track the location of the various location sensing electrodes within the localization volume.

In embodiments, the EAM system 70 is equipped for both magnetic and impedance tracking capabilities. In such embodiments, impedance tracking accuracy can, in some instances, be enhanced by first creating a map of the electric field induced by the electric field generator within the cardiac chamber of interest using a probe equipped with a magnetic location sensor, as is possible using the aforementioned RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 105 or another catheter or probe equipped with sensing electrodes, to generate, and display via the display 92, detailed three-dimensional geometric anatomical maps or representations of the cardiac chambers as well as electro-anatomical maps in which cardiac electrical activity of interest is superimposed on the geometric anatomical maps. Furthermore, the EAM system 70 may generate a graphical representation of the various tracked devices within the geometric anatomical map and/or the electro-anatomical map.

While the EAM system 70 is shown in combination with the electroporation catheter system 60 to provide a comprehensive depiction of an exemplary clinical setting 10, the EAM system 70 is not critical to the operation and functionality of the electroporation catheter system 60. That is, in embodiments, the electroporation catheter system 60 can be employed independently of the EAM system 70 or any comparable electro-anatomical mapping system.

In the illustrated embodiment, the electroporation catheter 105 includes a handle 105a, a shaft 105b, and an electroporation electrode arrangement 150, which is described further hereinafter. The handle 105a is configured to be operated by a user to position the electroporation electrode arrangement 150 at the desired anatomical location. The shaft 105b has a distal end 105c and generally defines a longitudinal axis of the electroporation catheter 105. As shown, the electroporation electrode arrangement 150 is located at or proximate the distal end 105c of the shaft 105b. In embodiments, the electroporation electrode arrangement 150 is electrically coupled to the electroporation generator 130, so as to receive electrical pulse sequences or pulse trains, thereby selectively generating electrical fields for ablating the target tissue by irreversible electroporation.

As described above and also in greater detail elsewhere herein, the electroporation catheter system 60 is operable to generate a multidirectional electric field for ablating target tissue via irreversible electroporation. Such procedures include single-shot ablation procedures, e.g., pulmonary vein isolation (PVI) procedures as well as focal cardiac ablation procedures.

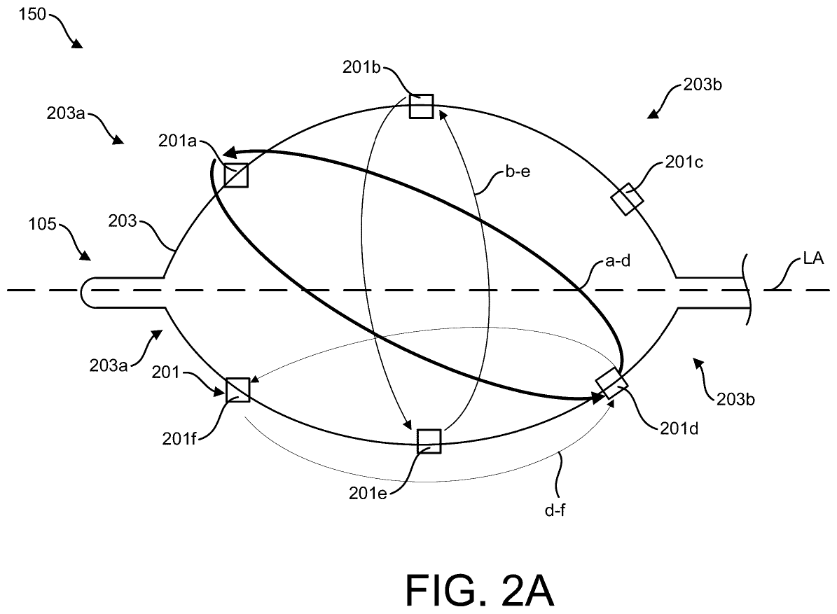

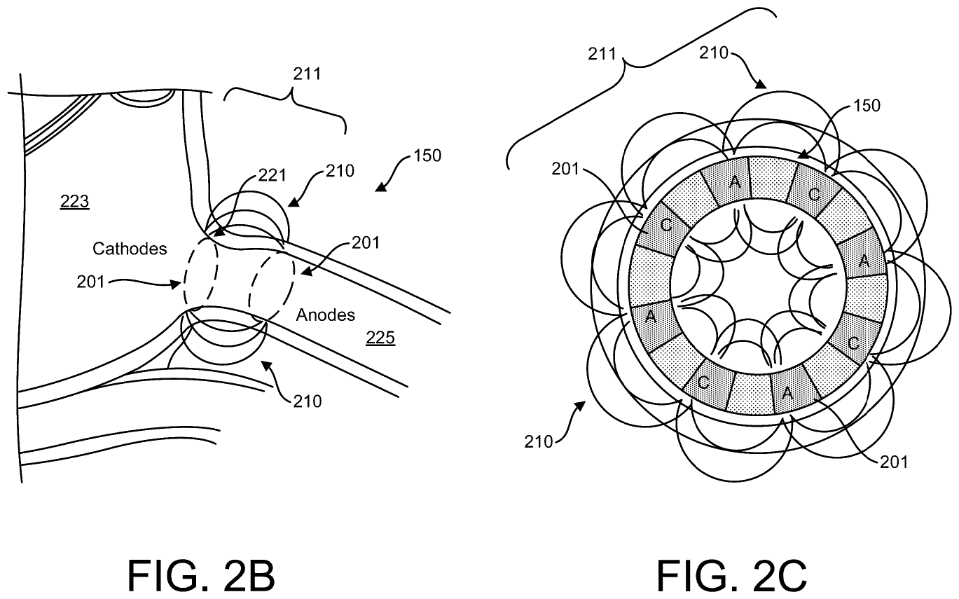

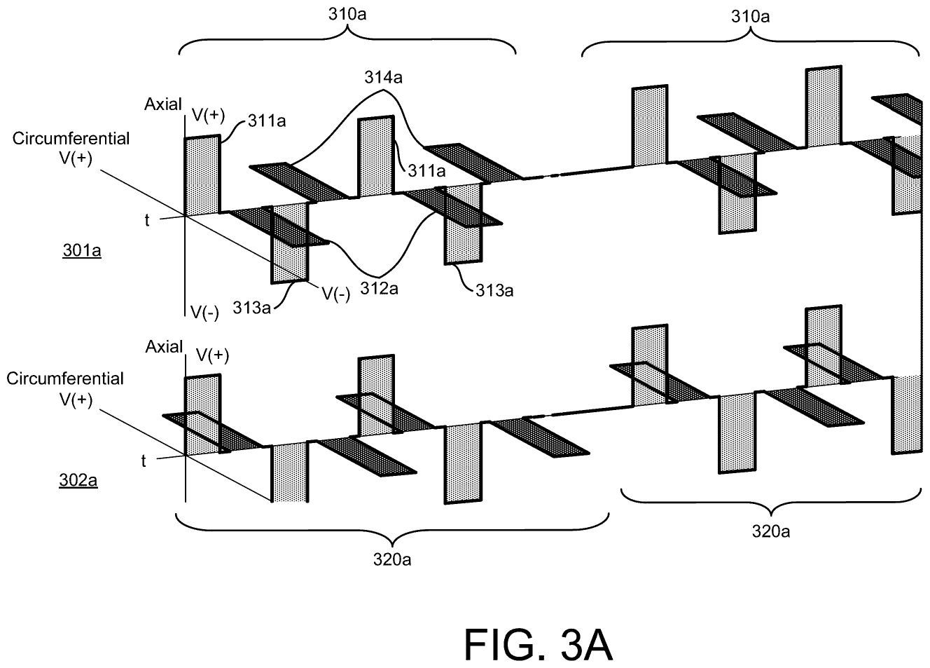

FIGS. 2A-2C show features of the electroporation catheter 105 that includes the electroporation electrode arrangement 150 according to embodiments. In the illustrated embodiment in FIG. 2A, the electroporation electrode arrangement 150 includes a plurality of electrodes 201a, 201b, 201c, 201d, 201e, and 201f arranged in a three-dimensional electrode array, such that respective ones of the electrodes 201a, 201b, 201c, 201d, 201e, and 201f are spaced from one another axially (i.e., in the direction of the longitudinal axis LA), circumferentially about the longitudinal axis LA and/or radially relative to the longitudinal axis LA. In embodiments, the electrodes 201a, 201b, 201c, 201d, 201e, and 201f are each individually, selectively addressable via the electroporation generator 130 (FIG. 1) so as to define a plurality of anode-cathode electrode pairs, each capable of receiving an electrical pulse sequence from the electroporation generator 130 and, consequently, creating an electric field capable of selectively ablating target tissue via irreversible electroporation. FIG. 2A schematically illustrates interactions (e.g., current flows forming electric fields) between electrode pairs formed between electrodes 201 (e.g., 201a, 201b, 201c, 201d, 201e, and 201f) included in the electroporation catheter 105. In this figure, interactions are shown as paired arrows (e.g., a-d, b-e, and d-f) indicating current flows between electrodes 201. And electrode pairs (e.g., 201a and 201d, 201b and 201e, and 201d and 201f) are shown with their respective current flows (e.g., a-d, b-e, and d-f) labeled.

FIG. 2B is a diagram illustrating electric fields 210 generated by interactions between electrode pairs in the electroporation catheter 105. In this figure, axially oriented electric fields 210 are shown positioned at an ostium 221 between the left atrium 223 and the left inferior pulmonary vein 225. In embodiments, the axially oriented electric fields 210 are produced by delivering electrical pulses to axially spaced anodes and cathodes.

FIG. 2C is also a diagram illustrating electric fields 210 generated by interactions between electrode pairs in the electroporation catheter 105. But here, the electric fields 210 are circumferentially oriented. In embodiments, the circumferentially oriented electric fields 210 are produced by delivering electrical pulses to circumferentially spaced anodes (“A”) and cathodes (“C”).

Between FIGS. 2A-2C, it is apparent that multiple electric fields 210 may be generated simultaneously and/or sequentially and in axial and circumferential orientations. For example, in embodiments, axially and circumferentially oriented electric fields 210 can be generated non-simultaneously in a pre-defined sequence by selectively controlling the timing of the delivery of the electric pulses to the respective electrodes 201. In addition, it is understood that intermittently generated electric fields 210 caused by staggered interactions between sets of electrode pairs and electric field orientations other than axial and circumferential are not beyond the scope of this disclosure and are indeed described in detail hereinafter.

As may be seen in FIG. 2A, the electroporation electrode arrangement 150 may include a plurality of individually addressable electrodes 201 (e.g., anodes or cathodes) arranged so as to selectively define a plurality of electrode pairs (e.g., anode-cathode pairs). Each anode-cathode pair may be configured to generate an electric field when a pulse sequence is delivered thereto. The plurality of anode-cathode pairs may include at least two of a first anode-cathode pair, a second anode-cathode pair, and a third anode-cathode pair. The first anode-cathode pair may be arranged so as to generate a first electric field oriented generally circumferentially relative to the longitudinal axis when a first pulse sequence is delivered thereto. The second anode-cathode pair may be arranged so as to generate a second electric field oriented generally in a same direction as the longitudinal axis when a second pulse sequence is delivered thereto. The third anode-cathode pair may be arranged so as to generate a third electric field oriented generally transverse to the longitudinal axis when a third pulse sequence is delivered thereto. In embodiments, any combination of the first, second, and third pulse sequences may be delivered simultaneously or intermittently and may take a variety of forms as described hereinafter.

In embodiments, the electroporation electrode arrangement 150 may be configured so as to structurally arrange the electrodes 201a, 201b, 201c, 201d, 201e, and 201f into a distally-located first region and a more proximally-located second region. As such, electrode pairs may be formed across various electrodes 201 in the electroporation electrode arrangement 150 between first and second regions. For example, the electrodes 201d and 201f may be configured to form an electrode pair. Similarly, the electrodes 201a and 201d or electrodes 201b and 201e or the combination thereof may be selected to form respective electrode pairs. Thus, the electrode pairs may comprise axially spaced electrodes, transversely spaced electrodes, or circumferentially spaced electrodes. Additionally, in embodiments, a given electrode (e.g., 201d) may serve as a common electrode in at least two electrode pairs to generate electric fields 210.

FIG. 2B shows a diagram of exemplary electric fields 210 that may be generated by the electroporation electrode arrangement 150. The electroporation electrode arrangement 150 may be configured to generate a multidirectional electric field 210 when at least one pulse sequence is delivered thereto. The multidirectional electric field 210 may include at least two of the following directions relative to the longitudinal axis: generally axial, circumferential, and transverse. As used herein, transverse may mean at any non-parallel angle relative to the longitudinal axis. As described elsewhere herein, the electroporation electrode arrangement 150 may be configured to operatively couple to an electroporation generator that is configured to generate the at least one pulse sequence. The electroporation electrode arrangement 150 may be configured to receive the at least one pulse sequence from the electroporation generator. Thus, the electroporation electrode arrangement 150 and the electroporation generator may be in operative communication with each other. In this disclosure, such communication may be used to generate electric fields 210 that are at least substantially gapless.

Undesired gaps in electric fields 210 generated by the electroporation electrode arrangement 150 may be limited or at least substantially eliminated. Such gaps may potentially lead to lesion gaps and therefore require multiple repositions of a catheter, for example. Overlapping electric fields 210 may at least substantially limit the number of such gaps. In embodiments, at least some the electric fields 210 generated in the first pulse sequence set may overlap at least partially with each other. For example, adjacent electric fields 210 (e.g., axial, transverse, and/or circumferential) in a combined electric field 211 may intersect one another so that there are limited to no gaps in the combined electric field 211. Overlapping may occur at or near the periphery of adjacent electric fields 210 or may occur over a preponderance or majority of one or more adjacent electric fields 210. In this disclosure, adjacent means neighboring electrodes 201 or electrodes 201 otherwise near each other. The electroporation generator may be configured to generate pulse sequences used in generating overlapping electric fields.

The configuration of the electroporation electrode arrangement 150 in the various embodiments may take on any form, whether now known or later developed, suitable for a three-dimensional electrode structure. In exemplary embodiments, the electroporation electrode arrangement 150 may be in the form of a splined basket catheter, with respective electrodes 201a, 201b, 201c, 201d, 201e, and 201f positioned on a plurality of splines in any manner known in the art. In embodiments, the electroporation electrode arrangement 150 can be formed on an expandable balloon, e.g., with electrodes formed on flexible circuit branches or individual traces disposed on the balloon surface. In other embodiments, the electroporation electrode arrangement 150 may be in the form of an expandable mesh. In short, the particular structure used to form the electroporation electrode arrangement 150 is not critical to the embodiments of the present disclosure.

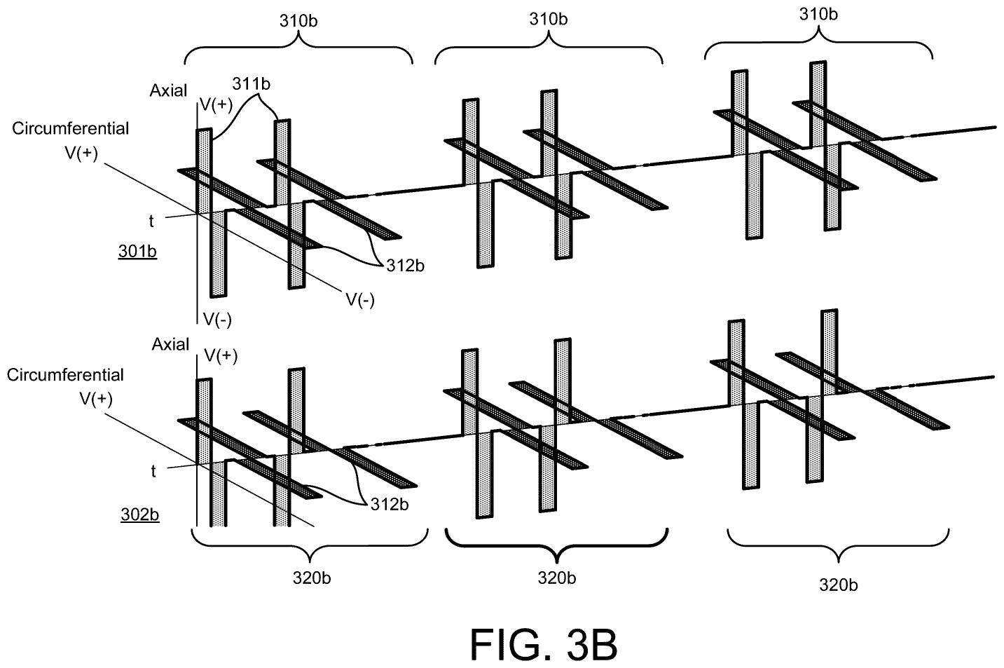

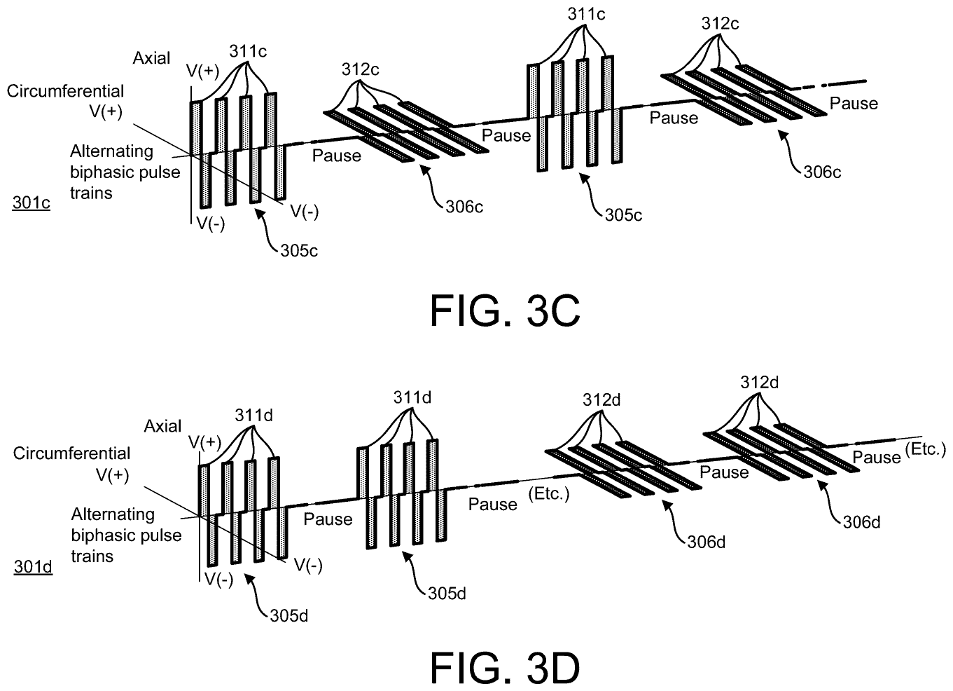

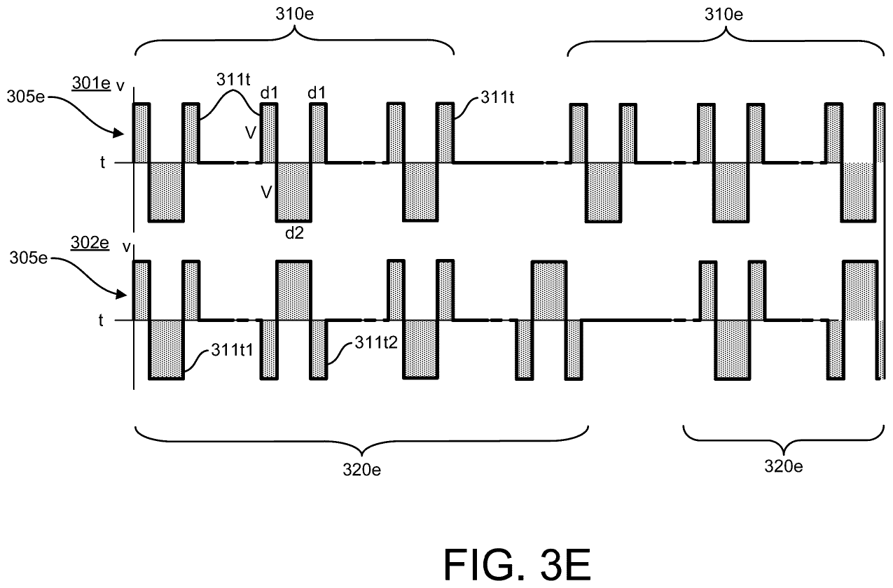

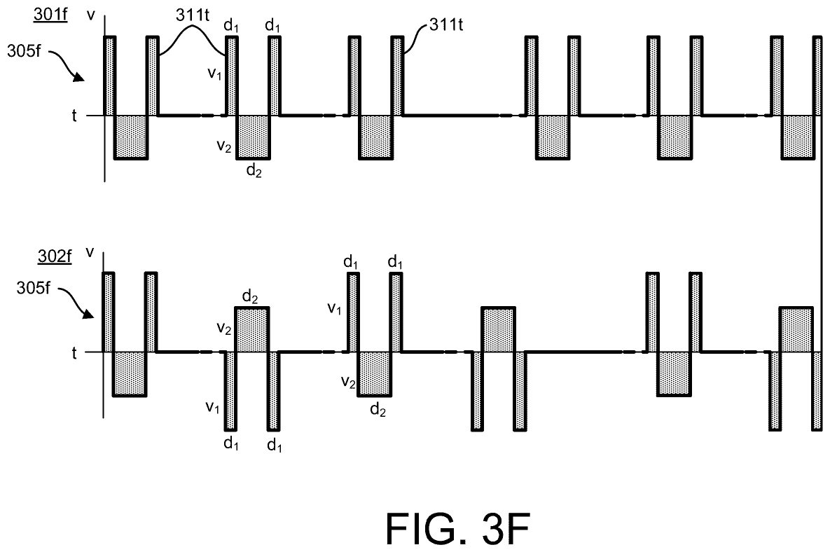

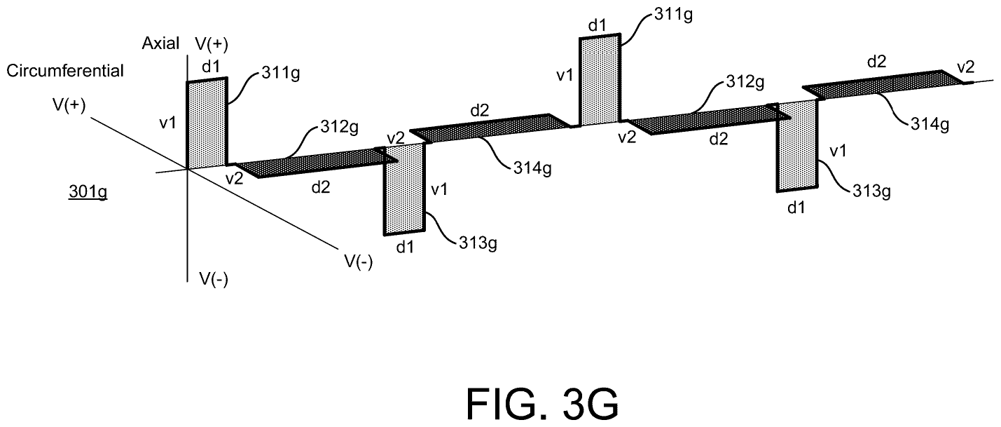

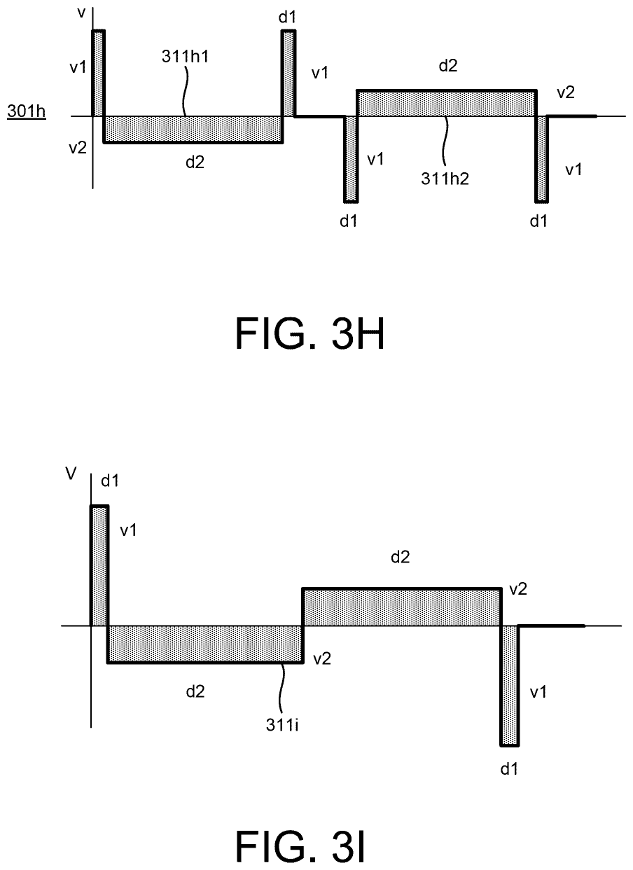

FIGS. 3A-31 show a variety of electrical pulse sequences that may be delivered to the electroporation electrode arrangement, e.g., by the electroporation generator, in accordance with embodiments of the subject matter of the disclosure. As can be seen in FIGS. 3A-31 and as described herein, the various exemplary pulse sequences that can be delivered to the plurality of anode-cathode pairs described above may include monophasic pulses biphasic pulses, triphasic pulses, and quadphasic pulses. Time-based characteristics of each pulse include an orientation (e.g., axial, circumferential, or transverse) and a polarity (e.g., positive (+) or negative (−)) as indicated on the axes.

FIG. 3A is a diagram illustrating two pulse sequence series 301a, 302a each having two pulse sequence sets 310a, 320a therein. As shown, the pulse sequence set 310a is comprised of monophasic direct current (DC) electrical pulses 311a, 312a, 313a and 314a. In the illustrated embodiment, the electrical pulses 311a, 313a are delivered to anode-cathode pairs oriented generally axially relative to the longitudinal axis of the electroporation catheter, whereas the electrical pulses 312a, 314a are delivered to anode-cathode pairs arranged circumferentially relative to the longitudinal axis. As illustrated, adjacent axially-directed electrical pulses 311a, 313a are monophasic DC pulses separated in time by a predetermined inter-pulse delay, and have an opposite polarity. Similarly, adjacent circumferentially-directed electrical pulses 312a, 314a are monophasic DC pulses separated in time by a predetermined inter-pulse delay, and have opposite polarity. It will be appreciated that in embodiments, the electrical pulses 311a and 313a may have the same (e.g., positive) polarity, and similarly, the electrical pulses 312a and 314a may also have the same polarity. As shown, a series of the electrical pulses 311a, 313a, 312a and 314a are grouped into two electrical pulse sequence sets 310a separated in time by a pause of predetermined length. In the illustrated embodiment, respective circumferentially-directed electrical pulses 312a, 314a are generated and delivered during an inter-pulse delay between adjacent axially-directed electrical pulses 311a, 313a.

In the illustrated embodiment, the pulse sequence series 302a and corresponding pulse sequence sets 320a differ from the pulse sequence series 301a and pulse sequence sets 310a in that the electrical pulses 312a, 314a are reversed in polarity.

With further reference to FIG. 3A, interlacing pulses within a pulse sequence set (e.g., 310a, 320a) may be accomplished using delays in pulse sequences within the pulse sequence set. As described elsewhere herein, the plurality of pulse sequence sets may have a first pulse sequence set 310a that includes both a first generated pulse sequence and a second generated pulse sequence. And each of the first generated pulse sequence (e.g., both pulses 311a) and the second generated pulse sequence (e.g., both pulses 312a) may include a plurality of pulses. The first generated pulse sequence may have a first inter-pulse delay between at least two pulses (e.g., between each pulse 311a in pulse sequence set 310a) therein. The first generated pulse sequence and the second generated pulse sequence may be alternatingly arranged within the first pulse sequence set 310a such that at least one pulse (e.g., the leftmost pulse 312a) in the second generated pulse sequence occurs within the first inter-pulse delay. The second generated pulse sequence may have a second inter-pulse delay between at least two pulses therein (e.g., between each pulse 312a in pulse sequence set 310a). At least one pulse in the first generated pulse sequence (e.g., the rightmost pulse 311a) may occur within the second inter-pulse delay. The plurality of pulse sequence sets may include a second pulse sequence set 310a, 320a that is separate from the first pulse sequence set 310a, 320a (respectively) and that has a pause between the first pulse sequence set and the second pulse sequence set. In embodiments, as shown here, the inter-pulse delay between any two pulse sequences (e.g., between 311a and 312a) may be less than the pause between any two pulse sequence sets. But this disclosure should not be limited to such a configuration because, for instance, in embodiments this inter-pulse delay may be less than or equal to the inter-pulse-set delay if desired. Delays may facilitate forming an interlaced pulse sequence set 310a and/or timing between pulse sequences. As one skilled in the art would appreciate, these concepts may extend across any number of pulses sequences or post sequence sets and any type of pulse.

It will be appreciated that although FIG. 3A depicts electrical pulses for delivery to axially and circumferentially-oriented electrode pairs, in other embodiments, one of these orientations may be substituted by transversely-oriented electrode pairs.

FIG. 3B is a diagram illustrating two pulse sequence series 301b, 302b each having two pulse sequence sets 310b, 320b that are each comprised of a plurality of pulse sequences with interlaced, biphasic DC pulses (e.g., 311b and 312b) to be delivered, respectively, to axially- and circumferentially-oriented electrode pairs in an electroporation electrode arrangement of the electroporation catheter. As shown, the pulse sequence sets 310b include axially-directed biphasic pulses 311b separated in time by an inter-pulse delay, and circumferentially-directed biphasic electrical pulses 312b temporarily interlaced with the axially-directed biphasic pulses. As further shown, in a given pulse sequence set 310b, the first circumferentially-directed electrical pulse 312b is generated during the inter-pulse delay between adjacent axially-directed electrical pulses 311b. It will be appreciated, however, that in other embodiments this order can be reversed. In the illustrated embodiment, the pulse sequence series 320b is substantially the same as the pulse sequence series 310b, except that in the pulse sequence series 320b the polarity of the circumferentially-directed electrical pulses 312b reverses from one pulse to the next.

With further reference to FIG. 3B, different sets of electrode pairs in the electroporation electrode arrangement may perform different pulse sequence series. A first set of electrode pairs may receive a first pulse sequence series 301b, and a second set of electrode pairs may receive a second pulse sequence series 302b. The first pulse sequence series 301b may be different from the second pulse sequence series 302b. In this way, for example, the electroporation electrode arrangement may have dedicated electrode pairs for generating electric fields in certain orientations. The first set of electrode pairs may generate one of an axially, transversely, or circumferentially orientated electric field, and the second set of electrode pairs may generate whichever of orientation the first set of electrodes did not. For example, as illustrated here, the first pulse sequence series 301b includes non-alternating pulses within pulse sequences (e.g., 312b within 301b) while the second pulse sequence series 302 includes alternating pulses within pulse sequences (e.g., 312b within 302b). The first and second pulse sequence series 301b, 302b may be synchronously or asynchronously generated and/or delivered to the electroporation electron arrangement. As described further hereinafter, if performed over time, these concepts may produce an electric field with dynamic gyration. Both the first and second set of electrode pairs may receive either the first or second pulse sequence series (e.g., 301b or 302b). In this way, the magnitude of the electric field in a given orientation may be increased over when only the first or second set of electrode pairs generates either the first or second pulse sequence series (e.g., 301b or 302b). Other pulse-manipulating techniques are helpful in increasing how effectively the electric field performs electroporation.

FIGS. 3C-3D are diagrams schematically illustrating alternative pulse sequence series 301c, 301d according to embodiments of the disclosure. As shown in FIG. 3C, the pulse sequence series 301c includes a plurality of alternating pulse sequences 305c, 306c separated in time by respective pauses. As shown, each pulse sequence 305c, 306c is made up of a series of biphasic DC electrical pulses 311c, 312c, respectively. In the illustrated embodiment, the pulse sequence 305c is delivered to axially-directed electrode pairs so as to generate a generally axially-oriented electric field, while the pulse sequence 306c is delivered to circumferentially-oriented electrode pairs so as to generate circumferentially-oriented electric fields. As discussed elsewhere herein, in embodiments, the aforementioned order can be reversed, and/or one of the pulse sequences 305c, 306c can be delivered to transversely-oriented electrode pairs.

In the embodiment of FIG. 3D, the pulse sequence series 301d comprises two electrical pulse sequences 305d separated by a predetermined pause, which are delivered to axially-oriented electrode pairs, followed by two electrical pulse sequences 306d separated by a predetermined pause and delivered to circumferentially-oriented electrode pairs. As with the embodiment of FIG. 3D, the aforementioned order can be reversed, and/or one of the pulse sequences 305d, 306d can be delivered to transversely-oriented electrode pairs.

It is emphasized that the disclosure should not be limited to the orientations shown in FIGS. 3A-3D. Although depicted in these figures as having axial and circumferential orientations, pulse sequences in this disclosure may include other combinations of orientations. For example, pulse sequences may include axial and transverse orientations, circumferential and transverse orientations, or each of axial, circumferential, and transverse orientations. Regardless of their form, pulse sequences may be arranged into pulse sequence sets and pulse sequence sets into pulse sequence series.

As discussed elsewhere herein, each of the generated pulse sequences delivered to the plurality of anode-cathode pairs may be charge-and-energy balanced, e.g., to prevent buildup of ionic byproducts and electrolysis and to help muscle stimulation. Although depicted and discussed with a certain number, orientation, or arrangement of pulse sequences, pulse sequence sets, or pulse sequence series, this disclosure should not be limited to such, as one skilled in the art would appreciate. As well, it is appreciated that pulse sequences may contain identical or different pulses and pulse sequence sets may contain identical or different pulse sequences.

FIG. 3E is a diagram illustrating two pulse sequences 305e, one non-alternating (301e) and the other alternating (302e), of charge-and-energy balanced, symmetric triphasic pulses (311t) to be delivered to electrodes in an electroporation electrode arrangement of the catheter. FIG. 3F. is a diagram illustrating two pulse sequences 305f, one non-alternating (301f) and the other alternating (302f), of charge-and-energy balanced, asymmetric triphasic pulses (311t) to be delivered to electrodes in an electroporation electrode arrangement of the catheter.

Referring to FIG. 3E, voltage shift and amplitudes may vary for a given pulse or between pulses according to embodiments of this disclosure. Rapid voltage shifts may open more pores in cell membranes. To produce this voltage shift, one or more pulses in a pulse sequence may have alternating polarity. For example, focusing on pulse sequence series 302e and pulse sequence set 310e therein, the one or more pulses may include a first pulse 311t1 and a second pulse 311t2. The second pulse 311t2 may be subsequent to the first pulse 311t1, and the first pulse 311t1 and the second pulse 311t2 may have alternating polarity. Triphasic pulses such as those shown in this figure create rapid voltage shifts within a pulse and may also do so between two pulses. For example, as shown, the first pulse 311t1 is a triphasic pulse that may alternate between positive, negative, and positive polarity, and the second pulse 311t2 is a triphasic pulse that alternates between negative, positive, and negative polarity. In embodiments, the first pulse 311t1 and second pulse 311t2 may be reversed without departing from the scope of this disclosure. As one skilled in the art would appreciate, these concepts may extend across any number of pulses. In addition, an increased number of open pores in cell membranes may be achieved in proportion to a high voltage amplitude, even for short pulse distances. Each of these concepts may be achieved while balancing charge and energy within the pulse sequence. The illustrated triphasic pulses have symmetric voltage amplitudes (v) and varied pulse lengths such that d1 is approximately equal to one half of d2 and are therefore charge-and-energy balanced. But depending on the desired application, these variables may proportionally vary to create higher voltage amplitudes with shorter pulse lengths and shorter voltage amplitudes with longer pulse lengths.

In embodiments, triphasic pulse sequences 305e, 305f may be delivered in a variety of forms while remaining charge-and-energy balanced. For example, boundary conditions may be established for each pulse and may include parameters for voltage amplitude and/or for pulse length. When the pulse sequences 305e, 305f delivered to the plurality of anode-cathode pairs comprise one or more triphasic pulses, each triphasic pulse may have a first voltage amplitude and a second voltage amplitude, the first voltage amplitude being greater than or equal to the second voltage amplitude. And each triphasic pulse may have a first voltage pulse length and a second voltage pulse length, the first voltage pulse length being less than or equal to the second voltage pulse length. The illustrated triphasic pulses have asymmetric voltage amplitudes (v) and varied pulse lengths such that d1 is less than d2 and are therefore charge-and-energy balanced while having a higher voltage amplitude than symmetric triphasic pulses (such as those shown in FIG. 3E). Regardless of form, pulse sequences such as those described above may be repeated during an electroporation operation to systematically open more pores in cell membranes.

In various embodiments, it may be advantageous to generate and selectively deliver to target tissue electrical pulse sequences that are not charge-balanced so as to promote electrolysis within the target tissue. Generally speaking, relatively short, high voltage electrical pulses are effective in causing reversible or irreversible electroporation in myocardial cells. In contrast, relatively long, low voltage electrical pulses can promote the formation of electrolytic byproducts in the myocardial tissue proximate the electrodes. These electrolytic byproducts can tend to diffuse outward along the electric field gradients and into the pores created in the cells via electroporation, thus causing, or at least encouraging, cell death due to an electrolytic imbalance within the cells. This technique can cause cell death in both irreversibly and reversibly electroporated cells. The present disclosure thus contemplates interlacing electrical pulses configured for causing irreversible electroporation with pulses configured for promoting the aforementioned electrolysis to enhance the likelihood of successfully ablating the target tissue.

FIG. 3G is a diagram schematically illustrating an exemplary pulse sequence series 301g comprised of interlaced axially-directed electrical pulses 311g, 313g, and circumferentially-directed electrical pulses 312g, 314g, with the electrical pulses 312g, 314g being interposed between adjacent electrical pulses 311g, 313g. As shown, the electrical pulses 311g, 313g are characterized by a pulse length d1 and a voltage V1, while the electrical pulses 312g, 314g are characterized by a pulse length d2 and a voltage V2. In embodiments, the pulse length d1 and the voltage V1 are relatively short and high, respectively, and are designed to ablate the target tissue by irreversible electroporation. Additionally, the pulse length d2 is substantially shorter than the pulse length d1, and the voltage V2 is substantially lower than the voltage V1. The resulting interlaced electric fields created at the respective electrode pairs to which the electrical pulses 311g, 312g, 313g and 314g are delivered can have the aforementioned dual effect of both ablating cells via irreversible electroporation and via cell death due to electrolytic imbalance.

FIGS. 3H and 31 schematically illustrate additional exemplary electrical pulse forms that can be employed in the various embodiments to promote ablation via both irreversible electroporation and cell death by creating an electrolytic imbalance in the target cells. FIG. 3H illustrates a pulse sequence series 301h comprised of a plurality of triphasic, charge imbalanced electrical pulses 311h1, 311h2 each having first and third phases of the same polarity characterized by a phase length d1 and a voltage V1, and a second phase characterized by a phase length d2 and a voltage V2. The electrical pulse 311h2 differs from the electrical pulse 311h1 in that the polarity of each phase is opposite that of the corresponding phase in the electrical pulse 311h1, although in other embodiments each corresponding phase can have the same polarity. As shown, in embodiments, the phase lengths d1, d2 and the voltages V1, V2 can be selected to result in an overall charge imbalance of the electrical pulse 311h1 or 311h2, with the phase length d1 and voltage V1 selected to cause electroporation and the phase length d2 and the voltage V2 selected to promote electrolysis and the formation of an electrolytic imbalance leading to cell death.

A similar effect can be achieved by employing the quad-phasic electrical pulse 311i illustrated schematically in FIG. 3I. As shown, the electrical pulse 311i is comprised of a first and fourth phase of opposite polarity and characterized by a phase length d1 and voltage V1, and second and third phases of opposite polarity each having the same phase length d2 and voltage V2. As shown, the phase length d1 is substantially shorter than the phase length d2, and the voltage V1 is substantially larger than the voltage V2, resulting in the aforementioned charge imbalance and corresponding dual-effect of causing cell death both by electroporation and electrolytic imbalance.

The electroporation generator may be configured to perform a feedback loop to loop the electroporation ablation system through a plurality of pulse sequences 305. In embodiments, the feedback loop may be automatic and/or may follow a programmed pattern. In either case, changes in orientation of the electric field may be continuous or intermittent. The feedback loop may be performed based on a target metric, such as electrogram amplitude reduction, impedance change, or another similar metric. When a target metric is achieved, for example, the electroporation generator may apply a generated pulse sequence across a different electrode pair than was previously used. As an example, the feedback loop may generate an axially oriented electric field followed by a transversely oriented electric field. It is appreciated that a feedback loop may also employ a mix of multidirectional and unidirectional electric fields in any order, and so this concept is not beyond the scope of this disclosure. Methods of this disclosure may make use of these concepts and of those described in relation to other embodiments disclosed herein.

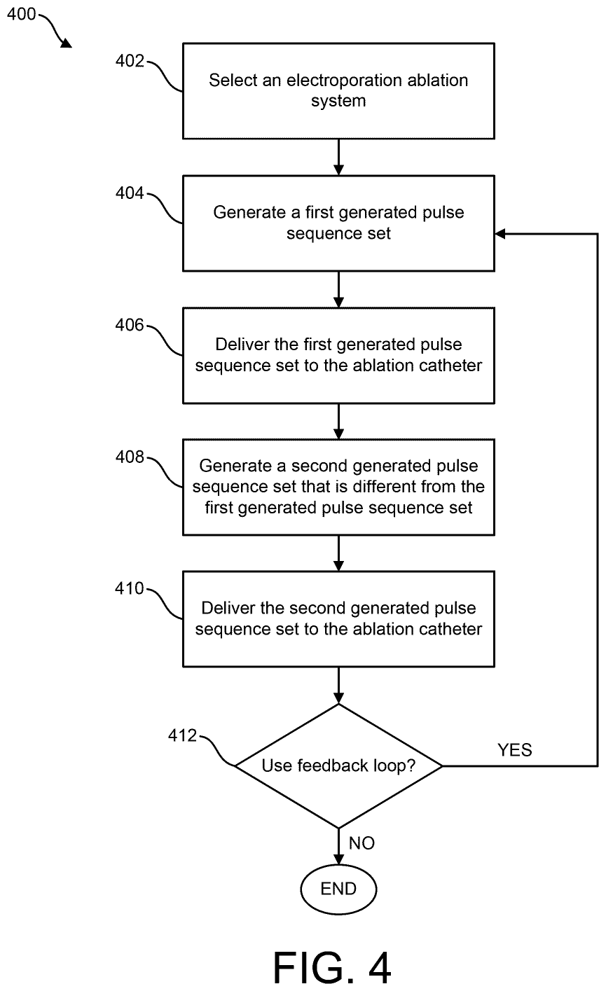

FIG. 4 is a diagram showing steps of a method 400 of generating and delivering a signal to electrodes in an electroporation ablation system. Such a method 400 and other related methods of generating and delivering a signal to electrodes in an electroporation ablation system are disclosed herein. Step 402 of the method 400 can include selecting an electroporation ablation system, which may be similar to those disclosed elsewhere herein. The method 400 may include generating, via the electroporation generator, a generated pulse sequence set including at least two of the first pulse sequence, the second pulse sequence, and the third pulse sequence. The method 400 may include delivering generated pulse sequence sets to the ablation catheter. Examples include generating a first generated pulse sequence set at step 404 and, at step 406, delivering the first generated pulse sequence set to the ablation catheter generating. At step 408, a second generated pulse sequence set may be generated and, at step 410, delivered to the ablation catheter. At step 412, the method 400 may include determining whether a feedback loop should be used as described hereinafter.

Generating a pulse sequence set can include generating a plurality of pulse sequence sets. At step 404, a first generated pulse sequence set may be generated, and at step 408, a second generated pulse sequence set that is different from the first generated pulse sequence set. For example, the first generated pulse set can be similar to those disclosed elsewhere herein, such as those with an axial orientation. And the second generated pulse set can be similar to those disclosed elsewhere herein, such as those with a circumferential orientation. In embodiments, generating the generated pulse sequence set may include alternatingly generating, over a time period, at least two of the first pulse sequence, the second pulse sequence, and the third pulse sequence to produce a dynamically gyrating electric field that comprises a changing pattern over the time period.

The gyrating electric field may be achieved via a feedback loop, such as those disclosed elsewhere herein. For example, at step 412, it may be determined whether to use a feedback loop. If so, the method 400 may loop back to a previous step such as step 404 to generate a first generated pulse sense set. And if no feedback loop is used at step 412, the method 400 may end.

In embodiments, delivering the generated pulse sequence set to the ablation catheter may employ sets of electrode pairs or depend on characteristic of the patient. For example, at step 408 a first generated pulse sequence may be applied across a first set of axially spaced electrodes, transversely spaced electrodes, or circumferentially spaced electrodes to produce a correspondingly oriented electric field. And at step 410 a second generated pulse sequence may be applied across whichever of the first set of axially spaced electrodes, transversely spaced electrodes, or circumferentially spaced electrodes that were not used in the first generated pulse sequence. In embodiments, the first and second generated pulse sequences may share at least one common electrode to which the first and second generated pulse sequences are applied. In embodiments, alternating waveforms may be delivered at different stages of the cardiac cycle (e.g., using local EGM sensing).

Various modifications and additions may be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An electroporation ablation system for treating target tissue, the electroporation ablation system comprising:
an ablation catheter including:
a handle;
a shaft having a distal end and defining a longitudinal axis of the ablation catheter; and
an electroporation electrode arrangement at the distal end of the shaft and including a plurality of electrodes spatially arranged so as generate a plurality of electric fields when an electrical pulse sequence is delivered to selected pairs of the electrodes, the plurality of electric fields including a first electric field having a first orientation relative to the longitudinal axis, and second electric field having a second orientation relative to the longitudinal axis; and
an electroporation generator operatively coupled to the electroporation electrode arrangement and configured to selectively generate and deliver the electrical pulse sequences to each selected pair of electrodes.

2. The electroporation ablation system of claim 1, wherein the first orientation is generally aligned with the longitudinal axis, and wherein the second orientation is circumferential about the longitudinal axis.

3. The electroporation ablation system of claim 1, wherein the first orientation is generally aligned with the longitudinal axis, and wherein the second orientation is transverse to the longitudinal axis.

4. The electroporation ablation system of claim 1, wherein the first orientation is transverse to the longitudinal axis, and the second orientation is generally circumferential about the longitudinal axis.

5. The electroporation ablation system of claim 1, wherein each of the plurality of electrical pulse sequences comprises a plurality of direct current (DC) pulses.

6. The electroporation ablation system of claim 5, wherein the DC pulses are monophasic pulses, biphasic pulses, or triphasic pulses.

7. The electroporation ablation system of claim 5, wherein the electroporation generator is configured to generate the DC pulses of each pulse sequence separated by an inter-pulse delay, and wherein at least some of the DC pulses of a first electrical pulse sequence are delivered during a respective inter-pulse delay of a second electrical pulse sequence.

8. The electroporation system of claim 1, wherein the electroporation generator is configured to generate a plurality of electrical pulse sequence sets having a pause therebetween, wherein each electrical pulse sequence set comprises a plurality of electrical pulse sequences.

9. The electroporation ablation system of claim 5, wherein the electroporation generator is configured to generate the DC pulses of each electrical pulse sequence separated by an inter-pulse delay, and wherein at least some of the DC pulses of the second electrical pulse sequence are delivered during a respective inter-pulse delay of the first electrical pulse sequence.

10. The electroporation ablation system of claim 5, wherein DC pulses are biphasic pulses, and the electroporation ablation generator is configured to alternate between delivering the first electrical pulse sequence to the first anode-cathode pair, and the second electrical pulse sequence to the second anode-cathode pair.

11. An electroporation ablation system for treating target tissue, the electroporation ablation system comprising:
an ablation catheter including:
a handle;
a shaft having a distal end and defining a longitudinal axis of the ablation catheter; and
an electroporation electrode arrangement at the distal end of the shaft and including a plurality of electrodes arranged so as to define a plurality of electrode pairs each configured to generate an electric field when an electrical pulse sequence is delivered thereto, the plurality of electrode pairs including:
a first electrode pair arranged so as to generate a first electric field having a first orientation relative to the longitudinal axis when a first electrical pulse sequence is delivered thereto; and
a second electrode pair arranged so as to generate a second electric field having a second orientation relative to the longitudinal axis when a second electrical pulse sequence is delivered thereto; and
an electroporation generator operatively coupled to the electroporation electrode arrangement and configured to selectively generate and deliver the first electrical pulse sequence to first electrode pair, and the second electrical pulse sequence to the second electrode pair.

12. The electroporation ablation system of claim 11, wherein the first orientation is generally aligned with the longitudinal axis, and wherein the second orientation is circumferential about the longitudinal axis.

13. The electroporation ablation system of claim 11, wherein the first orientation is generally aligned with the longitudinal axis, and wherein the second orientation is transverse to the longitudinal axis.

14. The electroporation ablation system of claim 11, wherein the first orientation is transverse to the longitudinal axis, and the second orientation is generally circumferential about the longitudinal axis.

15. The electroporation ablation system of claim 11, wherein the first and second electrical pulse sequences each comprise a plurality of biphasic DC pulses, and the wherein the electroporation ablation generator is configured to alternate between delivering the first electrical pulse sequence to the first electrode pair, and the second electrical pulse sequence to the second electrode pair.

16. A method of generating and delivering a signal to electrodes in an electroporation ablation system, the method comprising:
delivering a first electrical pulse sequence to a first electrode pair of an electroporation catheter having a longitudinal axis so as to generate a first electric field having a first orientation relative to the longitudinal axis; and
delivering a second electrical pulse sequence to a second electrode pair of the electroporation catheter so as to generate a second electric field having a second orientation relative to the longitudinal axis.

17. The method of claim 16, wherein delivering the first electrical pulse sequence to the first electrode pair and delivering the second electrical pulse sequence to the second electrode pair includes alternatingly delivering, over a time period, the first electrical pulse sequence to the first electrode pair and delivering the second electrical pulse sequence to the second electrode pair to produce a dynamically gyrating electric field that comprises a changing pattern over the time period.

18. The method of claim 17, wherein the first orientation is generally aligned with the longitudinal axis, and wherein the second orientation is circumferential about the longitudinal axis.

19. The method of claim 17, wherein the first orientation is generally aligned with the longitudinal axis, and wherein the second orientation is transverse to the longitudinal axis.

20. The method of claim 17, wherein the first orientation is transverse to the longitudinal axis, and the second orientation is generally circumferential about the longitudinal axis.
